(19) United States
(12) Patent Application Publication
(10) Pub. No.: US 2019/0200927 A1
(43) Pub. Date: Jul. 4, 2019

(54) DEVICES AND METHODS FOR DETECTING PENETRATION OF A SEMI-PERMEABLE MEMBRANE

(71) Applicant: The Trustees of Columbia University in the City of New York, New York, NY (US)

(72) Inventors: Anil K. Lalwani, New York, NY (US); Jeffrey W. Kysar, New York, NY (US); Hirobumi Watanabe, New York, NY (US)

(21) Appl. No.: 16/131,403

(22) Filed: Sep. 14, 2018

Related U.S. Application Data
(63) Continuation of application No. PCT/US17/22463, filed on Mar. 15, 2017.
(60) Provisional application No. 62/308,671, filed on Mar. 15, 2016.

(51) Int. Cl. A61B 5/00; A61B 5/06; A61M 37/00; A61F 11/00

(52) U.S. Cl. CPC A61B 5/685; A61B 5/065; A61M 2037/0023; A61F 11/00; A61M 2037/0061; A61M 37/0015

(57) ABSTRACT

A silver coated needle, a medical device comprising the needle and a method for electrochemically detecting penetration through a semi-permeable membrane such as the round window membrane of a subject are described. Penetration through the membrane by the needle can be detected by observing the voltage change as the needle penetrates through the membrane.

This application is a continuation of International Application No. PCT/2017/022463, filed Mar. 15, 2017 which claims priority to U.S. Provisional Patent Application, Ser. No. 62/308,671, filed Mar. 15, 2016, the contents of each of which are incorporated herein by reference thereto.

### BACKGROUND

Reliable methods allowing precise delivery of agents into the inner ear for therapeutic purposes, while preserving hearing function and maintaining cochlear architecture, remain a formidable challenge. Current options for intracochlear delivery are limited to systemic administration, transtympanic injection, or direct introduction into the cochlea. A drawback of systemic administration of a therapeutic agent is systemic toxicity. Transtympanic delivery has rapidly been incorporated into clinical practice but is hampered by variable efficacy and toxicity, as it relies on the diffusion properties of the round window membrane (RWM).

Direct introduction of the therapeutic agent into the cochlea can puncture the RWM. To prevent intracochlear trauma during the process of puncturing the RWM, it is important to limit the depth of microneedle insertion into the scala tympani. However, the exact moment of RWM perforation with the microneedle cannot always be visually ascertained, since direct visualization of the RWM can be surgically difficult. Thus, there is a need to accurately detect penetration of the RWM.

### SUMMARY

This disclosure relates to a needle useful for detecting perforation of a semi-permeable membrane.

In one aspect, the needle comprises a longitudinal metallic body comprising a bottom layer and a top layer, wherein the bottom layer comprises a base layer and the top layer comprises silver, wherein the needle is configured to operatively engage a voltage meter.

Example embodiments of the needle include any of the following non-limiting features.

Those wherein the base layer comprises copper; wherein the top layer comprises silver metal; wherein the metallic body comprises stainless steel, the base layer comprises copper and the top layer comprises silver; wherein the top layer includes silver chloride; and/or wherein the top layer has a thickness of about 2 μm (micro-meter).

The needle wherein at least a portion of the needle has an outer diameter of about 10 μm; wherein the metallic body has a taper from a proximal section to a distal section, wherein the proximal section has a greater outer diameter than the distal section; wherein the metallic body has a tip defined by the taper; wherein the metallic body has a proximal section having a first diameter, and a distal section having a second diameter; wherein the first diameter is greater than the second diameter; wherein a step is disposed between the first and second diameters.

The needle, wherein the metallic body comprises a hollow tube defining a channel for implanting a medical device, administering a therapeutic agent or sampling a fluid on the distal side of the membrane.

In another aspect, a medical device comprising the needle, including any of the non-limiting example embodiments of the needle enumerated above, is provided. Example embodiments of the medical device include those wherein the device is configured to advance the needle through a semi-permeable membrane.

Example embodiment of the device include those wherein the needle comprises a first electrode and a second electrode, wherein the medical device is configured to detect a voltage spike to determine an occurrence of penetration through the semi-permeable membrane by the needle. The voltage spike being detected when the needle contacts a sodium chloride-containing solution on the distal side of the semi-permeable membrane, and when the second electrode is in contact with a saline solution on the proximal side of the semi-permeable membrane, wherein the saline solution has a different concentration than the sodium chloride-containing solution, and the voltage spike comprises the difference in voltage between the sodium chloride-containing solution and the saline solution.

The medical device may comprise a voltage meter; such as wherein the voltage meter is electrically and operatively engaged with the needle and the second electrode and is configured to detect the voltage difference between the needle and the second electrode.

The medical device of any of the foregoing example embodiments may further comprise an actuator to advance the needle through the semi-permeable membrane; such as wherein the actuator is driven by an electric motor; including wherein the actuator is controlled by a servomechanism that stops the actuator when the voltage spike is detected.

The medical device of any of the previous embodiments may further comprise an electrical circuit to provide notification to an operator of the device that the semi-permeable membrane has been penetrated; including wherein the notification is audible, visual or a combination thereof.

The medical device of any of the foregoing example embodiments may be further configured to deliver a saline solution to the proximity of the semi-permeable membrane; and/or further configured to remove the saline solution from the proximity of the semi-permeable membrane.

The medical device of any of the foregoing example embodiments may comprise a distal end comprising the needle, the second electrode and a reservoir for a saline solution wherein the needle and the second electrode are in contact with the saline solution prior to advancement of the needle through the semi-permeable membrane. Example embodiments of the device include those wherein the distal end is configured to fit within the round window niche of a subject, and the semi-permeable membrane is the round window membrane; including those wherein the device is configured to deliver saline solution to the round window niche and/or wherein the device is configured to remove saline solution from the round window niche.

Also provided is a method for electrochemically detecting penetration through a semi-permeable membrane, the method comprising, for example, introducing a needle having a silver coated body section into the proximity of the semi-permeable membrane on its proximal side, wherein the needle is operatively engaged to a voltage meter and an electrode; advancing the needle to penetrate through the semi-permeable membrane; and detecting a voltage spike to determine an occurrence of penetration through the semi-permeable membrane.

Example embodiments of the method may further include the step of introducing saline solution to the proximal side of the semi-permeable membrane prior to advancing the needle; such as wherein the saline solution has a concentration of about 2 to about 5%.

In the method, the voltage spike can be detected when the needle contacts a sodium chloride-containing solution on the distal side of the semi-permeable membrane, and when the second electrode is in contact with a saline solution on the proximal side of the semi-permeable membrane. The saline solution having a different concentration than the sodium chloride-containing solution, and the voltage spike comprises the difference in voltage between the sodium chloride-containing solution and the saline solution.

Embodiments of any of the foregoing example embodiments of the method include those wherein the advancing of the needle is stopped when the needle penetrates through the semi-permeable membrane.

Embodiments of any of the foregoing example embodiments of the method include those wherein the needle is advanced through the semipermeable membrane by an actuator; such as wherein the actuator is driven by an electric motor; and/or wherein the actuator is controlled by a servomechanism that stops the actuator when the voltage spike is detected.

Any of the foregoing example embodiments of the method may further comprise notifying an operator that the semi-permeable membrane has been penetrated; such as wherein the notifying comprises providing a notification that is audible, visual or a combination thereof.

Any of the foregoing example embodiments of the method may further comprise removing the saline solution from the proximity of the semi-permeable membrane after the semi-permeable membrane has been penetrated.

Any of the foregoing example embodiments of the method may further comprise implanting a medical device, administering a therapeutic agent or sampling a fluid on the distal side of the membrane through the perforation of the membrane caused by the penetration of the membrane by the needle; such as wherein the needle is retracted from the perforation prior to implanting the medical device, administering the therapeutic agent or sampling the fluid; and or wherein the needle comprises a hollow tube providing a channel for implanting the medical device, administering the therapeutic agent or sampling the fluid.

According to an example embodiment of the method, the semipermeable membrane is the round window membrane of a subject. According to this example embodiment, a needle having a silver coated body section is introduced into a round window niche, wherein the needle is operatively engaged to a voltage meter, the needle is advanced into the niche to perforate the round window membrane, and a voltage spike is detected to determine an occurrence of penetration of the round window membrane.

Other example embodiments further include the step of introducing saline solution to the round window niche; such as wherein the saline solution is clinical saline; and/or wherein the saline has sodium chloride concentration of about 2 to about 5%.

The method may further comprise removing the saline solution from the round window niche after the round window membrane has been penetrated.

The method may further comprise implanting a medical device in the inner ear, administering a therapeutic agent or sampling the perilymph on the distal side of the round window membrane through the perforation of the membrane caused by the penetration of the membrane by the needle. In this embodiment, the needle may be retracted from the perforation prior to implanting the medical device, administering the therapeutic agent or sampling the perilymph; and/or wherein the needle comprises a hollow tube providing a channel for implanting the medical device, administering the therapeutic agent or sampling the perilymph.

Any of the aspects or embodiments of the method can be carried out using any of the embodiments of the needle and/or the medical device described above.

In another aspect, a method for electrochemically detecting perforation of a round window membrane is provided, the method comprising introducing a needle having a silver coated body section into a round window niche, wherein the needle is operatively engaged to a voltage meter, advancing the needle into the niche to perforate the round window membrane, and detecting a voltage spike to determine an occurrence of penetration of the round window membrane.

In some embodiments, the method further includes the step of introducing saline solution to the round window niche. In this regard, the saline solution is clinical saline. For example, the saline has sodium chloride concentration of about 2 to about 5%.

In other embodiments, the method further includes the step of introducing a therapeutic agent into the inner ear. The therapeutic agent can be introduced via a delivery channel in a hollow needle as described above. Alternatively, the therapeutic agent can be introduced through the perforation in the RWM left after the needle is withdrawn.

### BRIEF DESCRIPTION OF THE DRAWINGS

FIG. 1 illustrates a vertical Franz cell system according to an example embodiment.

FIG. 2 is a graph illustrating the detection of penetration of the round window membrane by observation of a voltage change according to an example embodiment.

FIG. 3 shows a graph depicting the voltage change upon membrane perforation with a device in accordance with the disclosed subject matter.

While the present disclosure is susceptible to various modifications and alternative forms, specific example embodiments have been shown in the figures and are herein described in more detail. It should be understood, however, that the description of specific example embodiments is not intended to limit the invention to the particular forms disclosed, but on the contrary, this disclosure is to cover all modifications and equivalents as illustrated, in part, by the appended claims.

### DESCRIPTION OF EXEMPLARY EMBODIMENTS

The round window is one of the two openings from the middle ear into the inner ear. The round window is situated below and a little behind the oval window, from which it is separated by a rounded elevation, the promontory. It is located at the bottom of a funnel-shaped depression, the round window niche.

The round window is sealed by the secondary tympanic membrane or round window membrane, which vibrates with opposite phase to vibrations entering the inner ear through the oval window. It allows fluid in the cochlea to move, which in turn ensures that hair cells of the basilar membrane will be stimulated and that audition will occur.

The round window membrane has a complex saddle point shape. The visible central portion is concave (curved inwards) toward the tympanic cavity and convex (curved outwards) toward the cochlea; but towards the edges, where it is hidden in the round window niche, it curves the other way. This membrane consists of three layers: an external, or mucous layer, derived from the mucous lining of the tympanic cavity; an internal layer, from the lining membrane of the cochlea; and an intermediate, or fibrous layer. The fibrous layer can be relatively difficult to penetrate, by for example a needle, so some force is needed to do so. Once the fibrous layer is penetrated, the abrupt change in resistance to the needle penetration can result in the needle being inserted beyond a desirable distance into the inner ear before the force can be reduced. This can result in damage or trauma to the inner ear. Therefore, it is desirable to determine when the needle has penetrated the RWM, so that force can be reduced and insertion of the needle into the inner ear stopped. Further, it is desirable that the surgeon can be notified promptly to reduce force applied to the needle.

The scala tympani is the lymph-filled spirally arranged canal in the bony canal of the cochlea that is separated from the cochlear duct by the basilar membrane, communicates at its upper end with the scala vestibuli, and abuts at its lower end on the membrane (the RWM) that separates the round window from the middle ear.

Perilymph (also known as Cotunnius' liquid, and liquor cotunnii) is an extracellular fluid located within the cochlea in two of its three compartments: the scala tympani and scala vestibuli. The ionic composition of perilymph is comparable to that of plasma and cerebrospinal fluid. The major cation in perilymph is sodium, with lesser amounts of potassium.

Silver Coated Needles

In one aspect, a needle useful for detecting perforation of a semi-permeable membrane is provided. In this disclosure, particularly, a microneedle that has the function of penetration of the RWM is described. In one embodiment, the needle has a longitudinal body having a proximal section and a distal section and opposing proximal and distal ends. The needle may have a solid metallic body or it may comprise a hollow tube. At least one section of the needle comprises silver, preferably the distal end. More particularly, the needle comprises a combination of silver and silver chloride. When a silver or silver chloride outer layer on the needle is used as an electrode interfacing a solution with chloride ions, the interface produces a voltage depending on the concentration of the chloride ions. The needle is configured to operatively engage a voltage meter, such that when it penetrates a semipermeable membrane, such as the round window membrane, a voltage spike is detected.

The needle is highly conductive and can function as a first electrode when in electrical and operational engagement with a voltage meter and a second electrode. The needle, the second electrode and the voltage meter can be used to detect small differences in voltage between solutions of differing chloride ion concentrations separated by a semipermeable membrane. When the needle and the second electrode are placed in the same solution containing chloride ions, the voltage meter will indicate that there is no voltage difference. To detect a voltage difference due to differing chloride ion concentration, the second electrode remains in contact with a reference saline solution of known concentration. The needle is passed through the semipermeable membrane into another solution with a different chloride ion concentration. The voltage meter will register a voltage change as the needle passes through the membrane from the reference solution to the other solution.

In one embodiment, the longitudinal body is a metallic tube having a base layer and an outermost layer of materials. The bottom layer comprises a base layer and the top layer comprises silver. The base layer can comprise copper and the top layer can comprise silver or silver chloride. In some embodiments, the metallic tube is stainless steel. In some embodiments, the top layer has a thickness of about 2 μm.

The longitudinal body, e.g., metallic tube, of the needle may have a taper from a proximal section to a distal section. In some embodiments, the longitudinal body can have a tip defined by the taper. The proximal section may have a first diameter, while the distal section has a second diameter. In this regard, the first diameter is preferably greater than the second diameter but not necessarily. In some instances, a step is disposed between the first and second diameters. The needle can have a tip diameter of about 10 μm.

A tapered needle may be useful in improving ease of insertion of the needle through the membrane, such as the RWM, by allowing a more gradual widening of the hole or perforation as the needle passes through it. A tapered needle may also be useful in allowing selection of the size of the opening in the membrane by selectively inserting the needle into the scala tympani to a depth that corresponds to a specific diameter of the needle.

A step disposed between the first and second diameters may allow easy detection of the depth of insertion of the needle into the scala tympani. The step will provide a stop on the needle that would require more force to penetrate the membrane than need for penetration of the needle tip. The resultant resistance to further penetration could signal the surgeon that the desired depth was reached.

According to an example embodiment, a stainless steel needle with the tip size as small as 10 micrometer coated with silver or silver chloride thin film is used to penetrate the RWM. A microneedle that aims at making a small hole benefits from this capability when a precise control in the size is necessary and too-deep penetration must be avoided.

Membrane Penetration and Perforation

Also provided is a method for electrochemically detecting penetration through a semi-permeable membrane, the method comprising, for example, introducing a needle having a silver coated body section into the proximity of the semi-permeable membrane on its proximal side, wherein the needle is operatively engaged to a voltage meter and an electrode; advancing the needle to penetrate through the semi-permeable membrane; and detecting a voltage spike to determine an occurrence of penetration through the semi-permeable membrane.

Example embodiments of the method may further include, for example, the step of introducing saline solution to the proximal side of the semi-permeable membrane prior to advancing the needle; such as wherein the saline solution has a concentration of about 2 to about 5%.

In the method, the voltage spike can be detected when the needle contacts a sodium chloride-containing solution on the distal side of the semi-permeable membrane, and when the second electrode is in contact with a saline solution on the proximal side of the semi-permeable membrane, wherein the saline solution includes a different concentration than the sodium chloride-containing solution, and the voltage spike comprises the difference in voltage between the sodium chloride-containing solution and the saline solution.

According to an example embodiment, a method for electrochemically detecting perforation of a round window membrane is provided, the method comprising introducing a needle having a silver coated body section into a round window niche, wherein the needle is operatively engaged to a voltage meter, advancing the needle into the niche to perforate the round window membrane, and detecting a voltage spike to determine an occurrence of penetration of the round window membrane.

According to example embodiments involving the RWM, the method may further include the step of introducing saline solution to the round window niche. In this regard, the saline solution is clinical saline. For example, the saline has sodium chloride concentration of about 2 to about 5%.

The method is generally described herein with reference to the RWM as an example of a semi-permeable membrane. The method is not limited to the RWM, but can be used with any semi-permeable membrane, including any membrane in an animal or human subject.

According to example embo, the chemical makeup of perilymph solution (that is high in sodium and low in potassium) is employed for detecting the precise moment of RWM perforation by a needle as it travels from the middle ear space, across the RWM, into the perilymph-containing scala tympani.

The concentration of NaCl in perilymph may be about 0.12 to about 0.14M, such as about 0.125M (125 mM), while the cation (Na⁺) to anion (Cl⁻) mobility ratio is about 1.52:1. By filling the round window niche with a known saline concentration, a chloride ion difference across the RWM can be created. A silver chloride plated needle as described herein can then be used to measure the voltage change as the needle transitions from a saline solution into perilymph. The theoretical voltage difference (diffusion potential plus electrode junction voltage) between two electrolytic solutions, separated by a semi-permeable membrane and measured by the same ion-containing electrodes, can be calculated using the following equation:

$${V}_{A} - {V}_{B} = \frac{{kB}^{T}}{e} ln ( \frac{{c}_{2}}{{c}_{1}} ) - \frac{{kB}^{T}}{e} ( \frac{{µ}_{+} - {µ}_{-}}{{µ}_{+} - {µ}_{-}} ) ln ( \frac{{c}_{1}}{{c}_{2}} ) \qquad ( 1 )$$

where V_{A}-V_{B} is the voltage difference between solutions, kB is the Boltzmann constant (8.617×10-5 eV/K), T is the temperature in Kelvin, and e is the number of electrons transferred between samples. The three constants simplify to 25.8 mV at room temperature. Thus, the equation depends on the ionic concentrations of the two solutions c1 and c2, as well as the relative ionic diffusion mobility of the cation and anion, u+ and u− respectively.

The method was demonstrated using artificial membranes to simulate the RWM and an artificial solution having a concentration similar to human perilymph in a divided cell as described below.

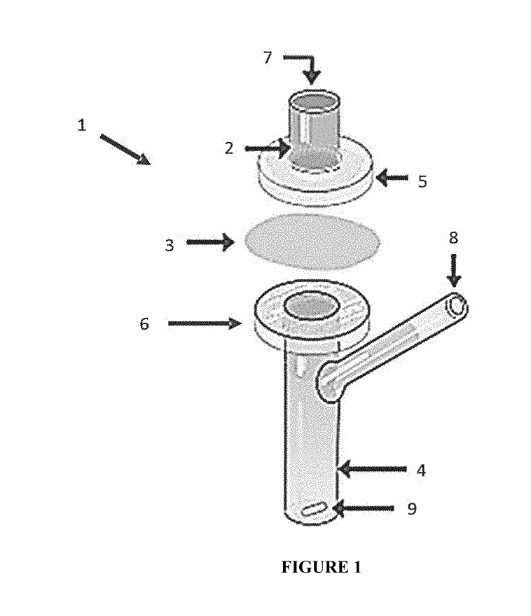

Voltage recordings were performed on 30 artificial membranes using a 3-mL vertical Franz™ Cell diffusion system (PermeGear, Inc., Hellertown, Pa.) as described in more detail in the Examples section herein. The cell 1 is illustrated in in exploded view in FIG. 1 (image used with permission by PermeGear). It consists of three main parts: a donor chamber 2, the artificial membrane 3, and a receptor chamber 4. The artificial membrane 3 was placed between upper flange 5 and lower flange 6, and a pinch clamp (not shown) held the flanges together to eliminate leaks. Donor port 7 and receptor port 8 allow solutions to be added to or sampled from the donor chamber 2 and receptor chamber 4, respectively. A stir bar 9 can be used during diffusion experiments to enhance mixing, but was not necessary for these experiments.

The donor chamber 2 at the top was filled with saline solution at concentrations varied from 1 to 5% and represented the middle ear space. The artificial membrane 3 separated the donor chamber 2 above from the receptor chamber 4 below akin to the RWM of the inner ear. The receptor chamber 4 on the bottom was filled with artificial perilymph to represent the inner ear space.

A needle as described herein and a second electrode were configured to be in operative connection to a voltage meter. Both the needle and the electrode were placed in the donor chamber 2 through the port 7. The needle was pushed vertically onto the membrane to penetrate or perforate it while the electrode remained in the donor chamber 7 and the voltage was monitored. Needle penetration was also observed visually.

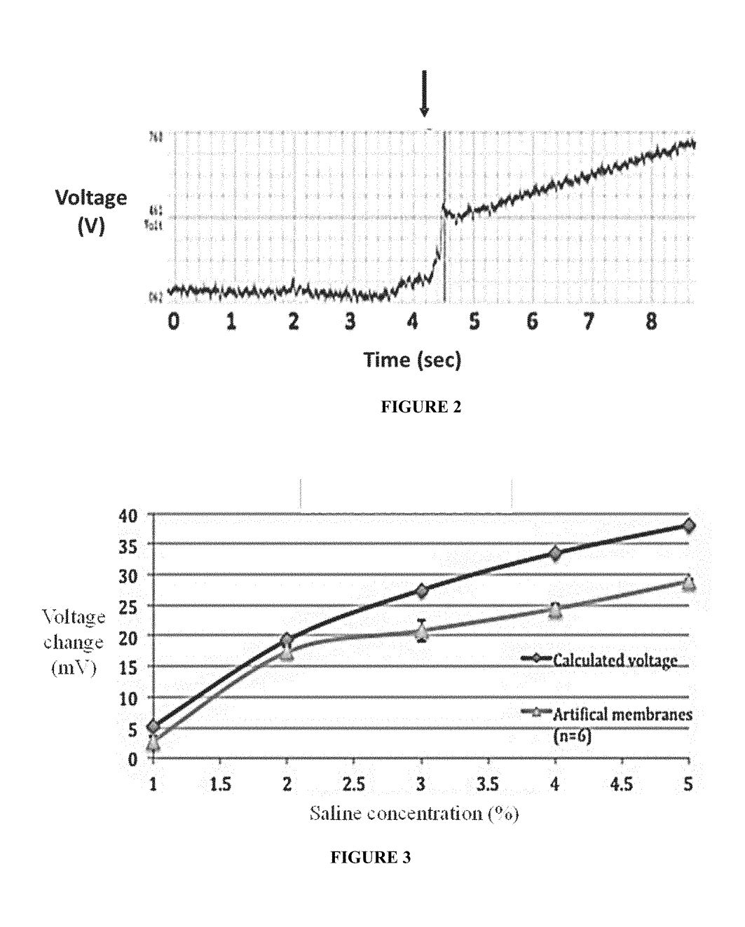

FIG. 2 shows a graph of voltage versus time during the process of membrane perforation. The voltage was measured with WinDaq voltage software. The initial “zeroed” part of the graph shows the voltage while both electrodes rested in the donor chamber, reaching approximately 0.62 V (after gain of 100). For all trials (n=30), there was a visible voltage change (spike) observed at the instant of membrane perforation by the silver chloride needle. An example of such a spike can be seen in FIG. 2. The spike was observed within milliseconds of the experimenter's visual and tactile confirmation of artificial membrane perforation, by both feeling the force needed for perforation compared to movement through solution as well as having both the vertical cell membrane and computer screen within one's field of vision. In this example, it is noted that after the initial spike, the voltage continued to increase gradually over the next few seconds and then leveled off as the needle entered the receptor chamber, but did not surpass the calculated voltage.

The voltage changes in the instant of penetrating into artificial perilymph solution were compiled into FIG. 3 and Table 1, showing the results from the trials of each saline concentration. The new voltage was measured as the value at which the steep spike changed slopes to level off, right near the 0.461 point in FIG. 2. FIG. 3 shows a graph of voltage change versus saline concentration, which are also summarized in Table 1. As shown in FIG. 3 and Table 1, the artificial membrane trials (triangles) were less than the calculated voltages (squares) for all saline concentrations from 1 to 5%. A direct relationship between saline concentration gradient and voltage was observed. The observed voltage spike was between 41.0% and 84.0% of the predicted value, with no trend in percentage versus saline concentration. However, the absolute voltage difference between experimental and theoretical values increased as the predicted (and measured) voltage changes increased, from 3.78 mV at 1% saline to 10.51 mV at 5% saline. The standard deviations of the six trials for 1 to 5% saline were 0.933, 0.923, 1.713, 0.757, and 0.422 mV, respectively.

TABLE 1
Predicted and Measured Voltage Change Upon Membrane
Perforation with Silver Plated Needles

| | 1% Saline | 2% Saline | 3% Saline | 4% Saline | 5% Saline |
|---|---|---|---|---|---|
| Calculated value (mV) | 6.41 | 20.62 | 28.79 | 34.8 | 39.4 |
| Ag plated needles (n = 6) (mV) | 2.63 ± 0.933 | 17.33 ± 0.923 | 20.79 ± 1.713 | 24.40 ± 0.757 | 28.89 ± 0.422 |

The magnitude of the voltage change was related to the saline concentration. However, the experimental voltage spike was less than the calculated value using Eq. (1). Without being bound by any particular hypothesis, the semipermeable membrane between the donor and receptor chamber may have allowed for diffusion of Na⁺ (sodium) and Cl⁻ (chlorine) ions during the time electrodes were being equilibrated prior to perforation. Mixing of the saline with the artificial perilymph solution upon puncture may have further reduced the electrolyte differences between the two solutions and reduced the magnitude of voltage spike. The mixing of solutions is also reflected in FIG. 2. It is believed, without being bound by any particular hypothesis, that the gradual increase in voltage after an initial voltage spike may be a reflection of the electrode sampling a purer portion of artificial perilymph as it delved deeper into the receptor chamber. Reasons for a decrease in voltage measurements may include small imperfections in the AgCl plating on the needle surface that are not visible to the human eye. There may also be a gradual loss of Cl⁻ from the needle over time once placed in the donor chamber.

In any event, a change in voltage was seen at all concentrations tested. The exact voltage measurement is not as critical as being able to detect a change denoting membrane perforation. Thus, voltage measurement reliably addressed the binary question of whether the membrane was “perforated or not perforated.”

In short, the testing illustrates that silver-plated needles can be used to confirm penetration of semipermeable membranes such as the RWM by detection of voltage change at the moment of perforation. The magnitude of the voltage change was related to the concentration of the saline solution; while the measured values were smaller than predicted, the moment of perforation could be electrically demonstrated across a wide range of saline concentration. Even with amplification, the background noise produced by the wires and the experimental environment was small relative to the voltages being measured during experimentation.

Desirably, advancing of the needle is stopped when the needle penetrates through the semi-permeable membrane such as the RWM to avoid damage to the structure(s) on the other side of the membrane.

The needle may be advanced through the semipermeable membrane by an actuator. The actuator may include, for example, a plunger in a syringe-like device that may be operated manually. Alternatively, the actuator may be driven by an electric motor. The actuator may be controlled by a servomechanism that stops the actuator when the voltage spike is detected.

As used herein, a servomechanism, or servo, is an automatic device that uses “error-sensing” negative feedback to control the action of a mechanism. A servo operates on the principle of negative feedback, where the control input is compared to the actual position of the mechanical system as measured by a transducer at the output. Any difference between the actual and wanted values (an “error signal”) is amplified (and converted) and used to drive the system in the direction necessary to reduce or eliminate the error.

In the method described herein, the negative feedback is triggered by the voltage spike detected by comparing the original voltage when the needle and the second electrode are both in the saline solution to the voltage after penetration of the needle through the membrane. When the voltage spike is detected, the feedback causes the actuator to stop advancing the needle further.

The method may further comprise notifying an operator that the semi-permeable membrane has been penetrated, such as wherein the notifying comprises providing a notification that is audible, visual or a combination thereof. The voltage detection circuitry and/or software could be configured to initiate a notification system once the membrane has been penetrated. Notification of the voltage change with perforation would provide the operator with an indicator to not insert the needle any deeper and thus avoiding damage to any structure(s) on the other side of the membrane.

For example, the circuitry and/or software could be configured to provide an audible tone such as a “beep” signaling membrane perforation. The notification could be a visual signal, such as a light indicating penetration. The voltage circuitry could also be configured to provide a display of the voltage detected during the method, which would include a visual indicator of the voltage spike. Combinations of one or more notifications could be used.

The method may further comprise removing the saline solution from the proximity of the semi-permeable membrane after the semi-permeable membrane has been penetrated. In a particular embodiment, the aline solution is removed from the round window niche after the RWM has been penetrated.

The method may further comprise implanting a medical device, administering a therapeutic agent or sampling a fluid on the distal side of the membrane through the perforation of the membrane caused by the penetration of the membrane by the needle; such as wherein the needle is retracted from the perforation prior to implanting the medical device, administering the therapeutic agent or sampling the fluid; and or wherein the needle comprises a hollow tube providing a channel for implanting the medical device, administering the therapeutic agent or sampling the fluid.

In other example embodiments, the method may further includes the step of, for example, introducing a medical device or a therapeutic agent into the inner ear, or sampling the perilymph. The medical device or therapeutic agent can be introduced via a delivery channel in a hollow needle as described above. Alternatively or additionally, the medical device or therapeutic agent can be introduced through the perforation in the RWM left after the needle is withdrawn, such as for sequential insertion of medical device(s) or repeated administration of a therapeutic agent. Sampling of the perilymph can be conducted through a hollow needle or through the perforation remaining after the needle is removed.

The experimental setup can easily be adapted for use clinically by simply placing saline solution adjacent to the RWM in the round window niche. The anatomy of the human middle ear and round window niche would allow a saline solution to rest in the space surrounding the RWM during electrode equilibration. Perforation of the RWM with a needle should only take a few seconds, so this time frame is sufficient to minimize inner ear electrolyte changes. The changes in K⁺ (potassium) and Na⁺ (sodium) activity within the scala tympani of guinea pigs in response to the middle ear space being flushed with saturated NaCl (sodium chloride) solution have been studied. It was found that increase in activity peaked at 30 min.

Before the penetration, slightly concentrated clinically available saline solution such as about 2 to 5%, compared to normal saline solution (0.9%), is provided around the small cavity called the round window niche in the middle ear space. In the case of a microneedle that aims at penetrating the RWM, the microneedle, acting as a first electrode, is connected to a voltage meter with another electrode placed near the round window niche such that these two electrodes can passively monitor the voltage. While the microneedle is in the air, large noise will be observed. When the microneedle makes contact with the concentrated saline solution, the noise will be gone and 0 voltage will be monitored. After the tip of the microneedle penetrates the RWM and is inside the inner ear space, a voltage of about 10 to about 25 mV will be observed. As soon as the rise in the voltage is monitored, the operator ceases advancing the microneedle. The concentrated saline solution in the round window niche can be washed away promptly. The microneedle can be retracted resulting in a perforation as small as the small tip of the microneedle.

Notification of the voltage change with perforation would provide the clinician with ample temporal warning to not insert the needle any deeper and thus avoiding inner ear trauma. The voltage software could also be configured to signal an audible notification system equipped with a device to provide an audible tone such as a “beep” signaling membrane perforation without requiring the clinician's eyes to leave the surgical field.

In a particular clinical situation, a surgeon or an operator can make a perforation in the RWM of a patient for implantation of a medical device, administration of therapeutic drugs or sampling of inner ear fluid.

The introduction of a microperforation in the RWM can significantly enhance intracochlear delivery of therapeutics. Therapeutic agents can diffuse much faster through the microperforation than through an intact RWM. Alternatively, a hollow microneedle can be used to deliver a therapeutic agent through the needle into the inner ear. Accordingly, in one aspect a method is provided for electrochemical detection of penetration by defining the precise instant a microneedle enters the inner ear.

Medical Device

A medical device comprising the silver containing needle, including any of the example embodiments of the needle enumerated above is described. Example embodiments of the medical device include those wherein the device is configured to advance the needle through a semi-permeable membrane. For example, a medical device for penetrating through the round window membrane and a method of detecting the entry of a medical device from middle ear space into inner ear space through the RWM are described.

Example embodiment of the device include those wherein the needle comprises a first electrode and a second electrode, wherein the medical device is configured to detect a voltage spike to determine an occurrence of penetration through the semi-permeable membrane by the needle; such as wherein the voltage spike is detected when the needle contacts a sodium chloride-containing solution on the distal side of the semi-permeable membrane, and when the second electrode is in contact with a saline solution on the proximal side of the semi-permeable membrane, wherein the saline solution has a different concentration than the sodium chloride-containing solution, and the voltage spike comprises the difference in voltage between the sodium chloride-containing solution and the saline solution.

The medical device may comprise a voltage meter; such as wherein the voltage meter is electrically and operatively engaged with the needle and the second electrode and is configured to detect the voltage difference between the needle and the second electrode.

The medical device may further comprise an actuator as described above to advance the needle through the semi-permeable membrane; such as wherein the actuator is driven by an electric motor; including wherein the actuator is controlled by a servomechanism that stops the actuator when the voltage spike is detected.

The medical device may further comprise an electrical circuit to provide notification to an operator of the device that the semi-permeable membrane has been penetrated; including wherein the notification is audible, visual or a combination thereof as described above.

The medical device may be further configured to deliver a saline solution to the proximity of the semi-permeable membrane; and/or further configured to remove the saline solution from the proximity of the semi-permeable membrane. In particular, the medical device may be configured to deliver and/or remove a saline solution from the round window niche.

The medical device may comprise a distal end comprising the needle, the second electrode and a reservoir for a saline solution wherein the needle and the second electrode are in contact with the saline solution prior to advancement of the needle through the semi-permeable membrane. Example embodiments of the device include those wherein the distal end is configured to fit within the round window niche of a subject, and the semi-permeable membrane is the round window membrane; including those wherein the device is configured to deliver saline solution to the round window niche and/or wherein the device is configured to remove saline solution from the round window niche.

The use of the needle, method and medical device described herein is not limited to the penetration of the RWM. Notably, the silver/silver chloride electrode embodied by the needle can detect the change in the concentration of the chloride ions around the electrode interface. Therefore, same method can be applied to an operation in which a precise perforation of a membrane is needed, such as in a cornea. The medical device can be adapted to other configurations by suitable modifications to enable penetration of membranes other than the RWM.

### EXAMPLES

These examples illustrate a simulation of penetrating the round window membrane using a silver-coated needle and measuring the voltage change as it penetrates the membrane.

Stainless steel Minutien pins, with a diameter of 0.2 mm (millimeter), were electroplated with copper, and then silver. Pins were then soaked in bleach for 24 hours to complete Ag/AgCl plating. Experiments were performed using a 3 mL (milliliter) Franz cell diffusion system with 1%, 2%, 3%, 4%, and 5% saline solution in the donor chamber and artificial perilymph solution in the receptor chamber separated by 5-μm pore synthetic membrane. Continuous voltage measurements were made throughout the process of membrane penetration by the needle (N=6 for each saline concentration).

Electroplating Procedures

All electroplating was performed in the Columbia University Department of Chemical Engineering, using a three-electrode system and a pAUTOLABIII potentiostat.

Copper Electroplating

With Ag/AgCl as the reference electrode and Pt as an auxiliary electrode, 0.2 mm diameter (20-μm tip) Minutien insect pins (Fine Science, Foster City, Calif.) were submerged in a bath of copper solution consisting of 0.63M Cu₂, 80 g/L H₂SO₄, 1.4 mM HCl, and 300 ppm polyethylene glycol (PEG). The copper was deposited to achieve a thickness of about 500 nm.

Silver Electroplating

After thorough washing of the copper-plated needles, the auxiliary electrode was replaced with a 0.999 pure Ag wire, and the bath with a silver nitrate solution (Caswell, Lyons, N.Y.). Silver was deposited to achieve a thickness of about 2 μm on the needle surface. Plated needles were then soaked in bleach for 24 h, to allow for the formation of a silver chloride layer.

The techniques described resulted in a qualitatively even, visible deposition of copper and silver layers on needles, while the pins maintained a pointed tip. The copper was an effective base layer for the silver-plating. Soaking samples in bleach for 24 h led to a visible darkening/graying of the silver needles from chloride deposition. This effect was not seen upon soaking in bleach for 1 to 4 h, while submersion for 72 h led to brittleness in the needles and a propensity for breaking during experimental set-up. Upon covering needle shafts in enamel, soldering to wire, and connection to an amplifier, silver chloride plating was confirmed by a zeroed voltage upon dipping both electrode tips in a saline solution. After the voltage reached about 0 mV, it remained within a close range of this value. Noise was minimal during this process, with oscillations of typically <1 mV prior to membrane perforation (around the range of the standard deviations below).

Franz™ Cell Experiments

Voltage recordings were performed on 30 synthetic membranes using a 3-mL vertical Franz™ Cell diffusion system (PermeGear, Inc., Hellertown, Pa.). Membranes as described below were placed between the flanges of the cell and secured with a pinch clamp. Solutions as described below were placed in the donor chamber and the receptor chamber.

Membrane.

Synthetic hydrophilic isopore membrane filters, 13 mm diameter with 5-lm pores were used in this study (EMD Millipore, Billerica, Mass.). An imperforate membrane was used for each trial.

Donor Chamber.

Stock sodium chloride solutions, 1% (0.171M), 2% (0.342M), 3% (0.513M), 4% (0.684M), and 5% (0.856M) by volume, were prepared using distilled water and solid NaCl. The donor chamber was filled with 0.3 mL of a saline solution with a given concentration. Six trials were recorded for each concentration, with fresh solution used for each perforation.

Receptor Chamber.

A stock of artificial perilymph was prepared using the following recipe: 125 mM NaCl, 3.5 mM KCl, 25 mM NaHCO₃, 1.2 mM MgCl2, 1.3 mM CaCl₂), and 0.75 mM NaH₂PO₄. The receptor chamber was filled with 3 mL perilymph solution, eliminating bubbles from the system visually and by tilting the Franz cell. The volume filling of the sampling port was adjusted to obtain the same height as the filled donor chamber.

The Ag or AgCl needles and reference electrode wires were painted with enameled nail polish for insulation, sparing the tip, then soldered to Teflon-insulated 20 gauge stranded steel wires to enhance conductivity. The electrodes were connected to a low noise, precision instrumentation amplifier with the gain set to 100 (model AMP01; Analog Devices, Norwood, Mass.). Voltages were recorded using a DI-718B Data Logger and WinDaqVR/Lite acquisition software (DATAQ Instruments, Inc., Akron, Ohio). For each trial, one needle and one AgCl wire reference electrode were placed in the donor chamber solution, and the voltage was measured until a reading of −0 mV was achieved. The data logger then recorded the voltage as the needle was manually lowered to penetrate through the synthetic membrane and contact perilymph solution in the receptor chamber. A spike in voltage illustrated on the acquisition software confirmed penetration. The needle was removed at the end of the 60 s.

Analysis

Voltage readings were recorded by the data logger every 0.083 s over the course of 60 s for each trial. Voltage was determined by measuring the differences in potential just prior to and immediately following perforation, to the moment at which the sharp spike decreased in slope or read a new constant level. Statistical analysis was performed with Microsoft Excel. All data are presented as their mean±standard deviation (SD).

The results show that silver-plated needles can detect an instantaneous change in voltage when traversing the membrane from saline solution into artificial perilymph. As calculated, the magnitude of the change in voltage upon penetration increased with increasing saline concentration and was stable across trials.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements.

Disclosed are the following aspects and embodiments.

One aspect provides a method for electrochemically detecting perforation of a round window membrane, the method comprising

introducing a needle having a silver coated body section into a round window niche, wherein the needle is operatively engaged to a voltage meter,

advancing the needle into the niche to perforate the round window membrane, and

detecting a voltage spike to determine an occurrence of penetration of the round window membrane.

Embodiments include those wherein the method further includes the step introducing saline solution to the round window niche; and wherein the saline solution has a concentration of about 2 to about 5%.

Embodiments of the method include any of the following aspects and embodiments related to the needle used in the method.

Those wherein the needle is silver chloride plated; wherein the needle includes a copper base layer; wherein the needle includes a layer of silver on top of the copper base layer; wherein the silver coating has a thickness of about 2 μm; wherein the needle has a stainless steel body; wherein the needle has a tip of about 10 μm; wherein the needle is a microneedle.

Another aspect provides a needle useful for detecting perforation of a semi-permeable membrane, the needle comprising:

a metallic tube having a base layer and an outermost layer of materials, including a bottom layer and a top layer, wherein the bottom layer comprises a base layer and the top layer comprises silver, wherein the needle is configured to operatively engage a voltage meter.

Embodiments of the needle include those wherein the base layer is copper; wherein the top layer comprises silver metal; wherein the metallic tube is stainless steel, the base layer is copper and the top layer comprises silver; wherein the top layer includes silver chloride; wherein the top layer has a thickness of about 2 μm; wherein at least a portion of the needle has a diameter of about 10 μm; wherein the metallic tube defines a hollow delivery channel for a therapeutic agent; wherein the metallic tube has a taper from a proximal section to a distal section; wherein the metallic tube has a tip defined by the taper; wherein the metallic body has a proximal section having a first diameter, and a distal section having a second diameter; wherein the first diameter is greater than the second diameter; and/or wherein a step is disposed between the first and second diameters.

While the disclosed subject matter is described herein in terms of certain exemplary embodiments, those skilled in the art will recognize that various modifications and improvements may be made to the disclosed subject matter without departing from the scope thereof. Moreover, although individual features of one embodiment of the disclosed subject matter may be discussed herein or shown in the drawings of the one embodiment and not in other embodiments, it should be apparent that individual features of one embodiment may be combined with one or more features of another embodiment or features from a plurality of embodiments.

1. A needle for detecting perforation of a semi-permeable membrane, comprising:
a longitudinal metallic body comprising a bottom layer and a top layer, the bottom layer comprises a base layer and the top layer comprises silver, the needle is configured to operatively engage a voltage meter.

2. The needle of claim 1, wherein the base layer comprises copper.

3. The needle of claim 1, wherein the top layer comprises silver metal.

4. The needle of claim 1, wherein the metallic body comprises stainless steel, the base layer comprises copper and the top layer comprises silver metal.

5. The needle of claim 1, wherein the top layer includes silver chloride.

6. The needle of claim 1, wherein the top layer has a thickness of about 2 μm.

7. The needle of claim 1, wherein at least a portion of the needle has an outer diameter of about 10 μm.

8. The needle of claim 1, wherein the metallic body includes a taper from a proximal section to a distal section, the proximal section includes an outer diameter great than an outer diameter of the distal section.

9. The needle of claim 8, wherein the metallic body includes a tip defined by the taper.

10. The needle of claim 1, wherein the metallic body includes proximal and distal sections, the proximal section includes a first diameter, and the distal section includes a second diameter.

11. The needle of claim 10, wherein the first diameter is greater than the second diameter.

12. The needle of claim 11, further comprising: a step disposed between the first and second diameters.

13. The needle of claim 1, wherein the metallic body comprises a hollow tube defining a channel for implanting a medical device.

14. A medical device comprising the needle of claim 1.

15. The medical device of claim 14, wherein the needle is configured to be advanced through a semi-permeable membrane.

16. The medical device of claim 15, wherein,
the needle comprises a first electrode, and the device further comprises a second electrode,
the medical device is configured to detect a voltage spike to determine an occurrence of penetration through the semi-permeable membrane by the needle.

17. The medical device of claim 16, wherein,
the voltage spike is detected upon (i) the needle contacting a sodium chloride-containing solution on the distal side of the semi-permeable membrane, and (ii) the second electrode is in contact with a saline solution on the proximal side of the semi-permeable membrane, and
the saline solution has a different concentration than the sodium chloride-containing solution, and the voltage spike comprises the difference in voltage between the sodium chloride-containing solution and the saline solution.

18. The medical device of claim 16, comprising a voltage meter.

19. The medical device of claim 18, wherein the voltage meter is electrically and operatively engaged with the needle and the second electrode, and the voltage meter is configured to detect the voltage difference between the needle and the second electrode.

20. The medical device of claim 1, further comprising an actuator to advance the needle through the semi-permeable membrane.

21. The medical device of claim 20, wherein the actuator is driven by an electric motor.

22. The medical device of claim 21, wherein the actuator is controlled by a servomechanism, the servomechanism is configured to stop the actuator upon detection of the voltage.

23. The medical device of claim 15, further comprising an electrical circuit configured to provide notification to an operator of the device that the semi-permeable membrane has been penetrated.

24. The medical device of claim 23, wherein the notification is audible, visual or a combination thereof.

25. The medical device of claim 15, wherein the needle is further configured to deliver a saline solution to the proximity of the semi-permeable membrane.

26. The medical device of claim 25, wherein the needle is further configured to remove the saline solution from the proximity of the semi-permeable membrane.

27. The medical device of claim 16, comprising a distal end comprising the needle, the second electrode and a reservoir for a saline solution, and
wherein the needle and the second electrode are in contact with the saline solution prior to advancement of the needle through the semi-permeable membrane.

28. The medical device of claim 27, wherein the distal end is configured to fit within the round window niche of a subject, and the semi-permeable membrane is the round window membrane.

29. The medical device of claim 28, wherein the medical device is configured to deliver saline solution to the round window niche.

30. The medical device of claim 29, wherein the medical device is configured to remove the saline solution from the round window niche.

31. A method for electrochemically detecting penetration through a semi-permeable membrane, the method comprising:
introducing a needle having a silver coated body section into the proximity of the semi-permeable membrane on its proximal side, the needle is operatively engaged to a voltage meter and an electrode,
advancing the needle to penetrate through the semi-permeable membrane, and
detecting a voltage spike to determine an occurrence of penetration through the semi-permeable membrane.

32. The method of claim 31, further comprising:
introducing saline solution to the proximal side of the semi-permeable membrane prior to advancing the needle.

33. The method of claim 32, wherein the saline solution includes a concentration of about 2 to about 5%.

34. The method of claim 31, wherein,
the voltage spike is detected upon (i) the needle contacting a sodium chloride-containing solution on the distal side of the semi-permeable membrane, and (ii) the second electrode is in contact with a saline solution on the proximal side of the semi-permeable membrane, and
the saline solution has a different concentration than the sodium chloride-containing solution, and the voltage spike comprises the difference in voltage between the sodium chloride-containing solution and the saline solution.

35. The method of claim 31, wherein the advancing of the needle is stopped when the needle penetrates through the semi-permeable membrane.

36. The method of claim 31, wherein the needle is advanced through the semipermeable membrane by an actuator.

37. The method of claim 36, wherein the actuator is driven by an electric motor.

38. The method of claim 36, wherein the actuator is controlled by a servomechanism that stops the actuator when the voltage spike is detected.

39. The method of claim 31, further comprising notifying an operator that the semi-permeable membrane has been penetrated.

40. The method of claim 39, wherein the notifying comprises providing a notification that is audible, visual or a combination thereof.

41. The method of claim 31, further comprising removing the saline solution from the proximity of the semi-permeable membrane after the semi-permeable membrane has been penetrated.

42. The method of claim 31, further comprising:
implanting a medical device; and
administering a therapeutic agent or sampling a fluid on the distal side of the membrane through the perforation of the membrane caused by the penetration of the membrane by the needle.

43. The method of claim 42, wherein the needle is retracted from the perforation prior to implanting the medical device.

44. The method of claim 42, wherein the needle comprises a hollow tube providing a channel for implanting the medical device, administering the therapeutic agent or sampling the fluid.

45. The method of claim 31, wherein the semipermeable membrane is the round window membrane of a subject.

46. The method of claim 45, further comprising:
introducing a needle having a silver coated body section into a round window niche, the needle is operatively engaged to a voltage meter;
advancing the needle into the niche to perforate the round window membrane; and
detecting a voltage spike to determine an occurrence of penetration of the round window membrane.

47. The method of claim 45, further comprising introducing saline solution to the round window niche.

48. The method of claim 47, wherein the saline solution is clinical saline.

49. The method of claim 48, wherein the saline has sodium chloride concentration of about 2 to about 5%.

50. The method of claim 47, further comprising removing the saline solution from the round window niche after the round window membrane has been penetrated.

51. The method of claim 31, further comprising:
implanting a medical device in the inner ear; and
administering a therapeutic agent or sampling the perilymph on the distal side of the round window membrane through the perforation of the membrane caused by the penetration of the membrane by the needle.

52. The method of claim 51, wherein the needle is retracted from the perforation prior to implanting the medical device.

53. The method of claim 51, wherein the needle comprises a hollow tube providing a channel for implanting the medical device.
